(19) United States
(12) Patent Application Publication
(10) Pub. No.: US 2011/0217743 A1
(43) Pub. Date: Sep. 8, 2011

(54) Method of Producing Lauric Acid-containing Oil or Fat

(75) Inventors: Hiroshi Yoshida, Cincinnati, OH (US); Fumikazu Takahashi, Tochigi (JP); Yasushi Takimura, Tochigi (JP)

(73) Assignee: Kao Corporation, Tokyo (JP)

(21) Appl. No.: 12/716,608

(22) Filed: Mar. 3, 2010

(51) Int. Cl. C12P 7/64

(52) U.S. Cl. 435/134

(57) ABSTRACT

To provide a method for supplying lauric acid with algae.

A method for producing an oil or fat containing 3 weight % or higher lauric acid in the fatty acid composition, by culturing an alga belonging to the genus Symbiodinium in a medium and recovering from the culture.

### FIELD OF THE INVENTION

The present invention relates to a method for producing an oil or fat containing lauric acid as a constituent fatty acid (hereinafter may also be referred to simply as “lauric acid-containing oil or fat”), the method employing an alga.

### BACKGROUND OF THE INVENTION

Lauric acid is a typical fatty acid contained in a large amount in coconut oil and palm kernel oil and is used as a raw material of a variety of surfactants, in foods, and for other materials.

Currently, the supply source of lauric acid is limited to coconut and palm kernels, which are grown in limited areas in the world. Cultivated lands now allocated to production of such lauric acid sources will be shared competitively with areas for bio-fuel for diesel engines and for food production. Excessive land cultivation for the production of lauric acid sources causes destruction of tropical rain forests.

Therefore, there is demand for creating a technique for supplying lauric acid, which technique does not rely on coconut or palm kernels.

Meanwhile, dinophyceae Crypthecodinium chonii, which grows not via photosynthesis but via heterotrophy, is known to be a lauric acid-producing organism and to have high lauric acid content (15.7%/total lipid) (Phytochemistry, (1988) 27, 1679-1683).

From the viewpoints of cost for carbon sources and other factors, more preferred are algae species which can grow via photosynthesis (autotrophy) and have higher lauric acid content. However, among such photoautotrophic algae species, only Neochloris oleoabundans, having a lauric acid content of about 1 to 2% at best, is known (J. Ind. Microbiol. Biotechnol. (2009) 36: 821-826), and no algae species has heretofore been known to have higher lauric acid content.

### SUMMARY OF THE INVENTION

The present invention relates to a method for producing an oil or fat containing lauric acid as a constituent fatty acid, which method includes culturing an alga belonging to the genus Symbiodinium in a medium and recovering, from the culture product, an oil or fat having a lauric acid content of 3 weight % or higher of the fatty acid composition.

The present invention also relates to a method for producing lauric acid, which method includes separating and recovering lauric acid from the oil or fat.

### MODES FOR CARRYING OUT THE INVENTION

The present invention provides a method for supplying lauric acid through employment of algae.

The present inventors have carried out studies on lauric acid-producing organisms, and have found that algae belonging to the genus Symbiodinium, which are a photoautotrophic dinophyceae, have high lauric acid content, and that an oil or fat containing lauric acid as a constituent fatty acid at high content can be efficiently produced by use of the algae.

According to the method of the present invention, which employs algae that can readily grow, an oil or fat containing lauric acid as a constituent fatty acid at high content can be efficiently produced, without imposing limitation on the cultivated fields for the growth of coconut and palm kernels or competing in the cultivated land with areas for food production, etc. In addition, according to the method of the present invention, destruction of tropical rain forests can be avoided.

The method of the present invention for producing a lauric acid-containing oil or fat includes culturing an alga belonging to the genus Symbiodinium in a medium and recovering, from the culture product, an oil or fat having a lauric acid content of 3 mass % or higher in the fatty acid composition.

The oil or fat has a lauric acid content of 3 weight % or higher of the fatty acid composition. The lauric acid content is preferably 5 to 60 weight %, more preferably 10 to 60 weight %.

The algae employed in the present invention may be any algae strains belonging to genus Symbiodinium in the class Dinophyceae, so long as the strains have an ability to produce an oil or fat having a lauric acid content of 3 weight % or higher in the fatty acid composition.

The algae of the present invention may be selected through, for example, the following screening procedure:

i) dispensing a sterilized medium (WA medium (see Table 2) as a fresh water medium or Daigo IMK medium (see Table 3) as a seawater medium) into a culture container;

ii) inoculating an alga strain to the medium and performing stationary culturing at room temperature (22° C. to 24° C.) under illumination (illuminance: about 3,000 lux, illumination for 12 hours and dark for 12 hours);

iii) recovering the produced algae and extracting oil or fat; methyl esterifying the fatty acids; and determining the fatty acid composition, to thereby select an alga strain which can produce a lauric acid-containing oil or fat; and

iv) selecting an alga strain having a lauric acid content of 3 weight % or higher based on the total fatty acid in the oil or fat.

Examples of preferred algae species include Symbiodinium microadriaticum, Symbiodinium goreaui, Symbiodinium linucheae, Symbiodinium bermudense, Symbiodinium meandrinae, Symbiodinium californium, Symbiodinium kawagutii, Symbiodinium corculorum, Symbiodinium consortia, Symbiodinium muscatinei, Symbiodinium freudenthal, Symbiodinium pulchrorum, Symbiodinium pilosum, and Symbiodinium sp. In some cases, Symbiodinium microadriaticum is called or described as Zooxanthella microadriatica or Gymnodinium microadriaticum. Similarly, Symbiodinium linuchae is in some cases called or described as Gymnodinium linuchae. Examples of more preferred Symbiodinium microadriaticum species include Zooxanthella microadriatica strain LB2281 (available from the Culture Collection of Algae at University of Texas at Austin (UTEX)) and strains having virtually the same phycological properties as those of strain LB2281.

The strain LB2281 has the following phycological properties. Strains belonging to the same genus as that of strain LB2281, and strains having virtually the same mycological properties as those of strain LB2281 can be identified on the basis of the following properties.

### <Phycological Properties of Strain LB2281>

i) symbiotic in other Protista, Mullsc, Coelenterata and the like, such as Foraminifera, Radiolaria, shellfish coral and Actiniaria.

ii) having flagella extending from the belly of a cell and cingula; and

iii) cell coat having no platy structure (thecal plate).

The algae of the present invention also encompass mutants of the aforementioned strain LB2281 or strains having virtually the same mycological properties as those of strain LB2281.

For example, a mutant strain designed so as to produce an oil or fat having a higher lauric acid content as compared with a corresponding wild-type strain is also included in the algae of the present invention.

The algae of the present invention belonging to genus Symbiodinium may be cultured in an appropriate medium prepared from natural or artificial seawater under illumination through a cultivation method generally employed in culturing of micro-algae.

The medium which may be employed in the invention is a known medium which contains natural or artificial seawater as a base, and additives such as a nitrogen source, a phosphorus source, a metal salt, and vitamins.

Examples of the nitrogen source include NaNO₃, KNO₃, Ca(NO₃)₂, NH₄NO₃, and (NH₄)₂SO₄. Examples of the phosphorus source include K₂HPO₄, KH₂PO₄, Na₂HPO₄, NaH₂PO₄, and sodium glycerophosphate. Examples of the metal salt include NaCl, KCl, CaCl₂, MgCl₂, Na₂SO₄, K₂SO₄, MgSO₄, Na₂CO₃, NaHCO₃, Na₂SiO₃, H₃BO₃, MnCl₂, MnSO₄, FeCl₃, FeSO₄, CoCl₂, ZnSO₄, CuSO₄, and Na₂MoO₄. Examples of the vitamins include biotin, vitamin B12, thiamine-HCl, nicotinic acid, inositol, folic acid, and thymine.

The aforementioned medium may further contain an appropriate additive such as a carbon source or a trace metal, in order to promote production of lauric acid-containing oil or fat.

Examples of preferred media include Daigo IMK medium, f/2 medium, ESM medium, Li medium, and MNK medium.

Preferably, the pH of the thus-prepared medium is adjusted to fall within a range of 7.0 to 8.0 through addition of an appropriate acid or base, and is sterilized in an autoclave before use.

In culturing, no particular limitation is imposed on the amount of algae inoculated to the culture medium. However, the amount is preferably 1.0 to 10.0% (vol/vol), more preferably 1.0 to 5.0% (vol/vol), with respect to the amount of culturing medium.

No particular limitation is imposed on the culture temperature, so long as the growth of the algae of the present invention is not adversely affected. Generally, the culturing is preferably performed at 10 to 30° C., more preferably 15 to 25° C.

Light irradiation may be performed under any conditions, so long as photosynthesis can be performed. Needless to say, either artificial light or sunlight may be employed.

The illuminance preferably falls within a range of 100 to 50,000 lux, more preferably 300 to 10,000 lux.

The pH during culturing is generally 6.5 to 8.5, preferably 7.0 to 8.0.

Culturing is performed so that an alga is grown in a high density. For example, the culturing period is 7 to 120 days, preferably 7 to 30 days. Any of aeration and agitation culturing, shake culturing, and stationary culturing may be employed.

After completion of culturing, an alga is separated through a customary method such as centrifugation or filtration. The thus-separated algae mass as is, or a broken product thereof obtained through sonication, by means of Dyno Mill or by other means is subjected to solvent extraction with organic solvent such as chloroform, hexane, butanol, methanol, or ethyl acetate, whereby lauric-acid-containing oil or fat can be recovered.

When strain LB2281 is used, 100 g of the dry algae contain a lauric acid-containing oil or fat in an amount of about 10 to about 20 g. That is, the amount of lauric acid-containing oil or fat produced in 1 L of medium reaches about 0.05 to about 0.1 g.

In this case, the oil or fat has a lauric acid content as high as 6.0 to 15.0 weight % of the fatty acid composition. Thus, the amount of produced lauric acid-containing oil or fat in 1 L of medium is as high as about 0.003 to about 0.015 g.

Lauric acid may be separated from the lauric acid-containing oil or fat by transforming the oil or fat into a fatty acid mixture or an ester of a fatty acid through a known method; and recovering high concentration of lauric acid through the urea addition method, cooling separation, HPLC, supercritical liquid chromatography, etc.

### EXAMPLES

### Referential Example 1

### Culturing of Algae and Analysis of Fatty Acid Composition

From the Culture Collection of Algae at University of Texas at Austin (UTEX), the following 20 algae strains were obtained.

TABLE 1
Algae strains and culture media

| Algae No. | Name | Class | Medium |
|---|---|---|---|
| LB 2658 | Chaetoceros gracilis | Bacillariophyceae | IMK |
| LB 2611 | Cyclotella meneghiniana | | IMK |
| B 2085 | Cylindrotheca fusiformis | | IMK |
| B 2049 | Stauroneis amphoroides | | IMK |
| 674 | Navicula pelliculosa | | WA |
| B 2047 | Nitzschia laevis | | IMK |
| 646 | Phaeodactylum tricornutum | | IMK |
| B 679 | Pinnularia sp. | | IMK |
| 1246 | Bracteacoccus grandis | Chlorophyceae | WA |
| LB 835 | Carteria radiosa | | WA |
| LB 2544 | Characium acuminatum | | WA |
| LB 2068 | Chlorella sp. | | IMK |
| LB 1970 | Chloromonas clathrata | | WA |
| 1783 | Chlorococcum refringens | | WA |
| LB 2423 | Cryptomonas sp. | Cryptophyceae | IMK |
| LB 2061 | Microcystis aeruginosa | Cyanophyceae | WA |
| LB 2425 | Phormidium persicinum | | IMK |
| LB 1636 | Ectocarpus variabilis | Phaeophyceae | IMK |
| LB 1426 | Sphacelaria cirrosa | | IMK |
| 157 | Botrydium cystosum | Xanthophyceae | WA |
| LB 2067 | Vaucheria bursata | | WA |

Culturing of algae was performed in the following methods. WA medium (composition, see Table 2) and a commercial medium (Daigo IMK medium, product of Nihon Pharmaceutical Co., Ltd.) (composition, see Table 3) were employed as a fresh water medium and a seawater medium, respectively.

TABLE 2
Composition of WA medium

| | for 1 L | |
|---|---|---|
| NaNO₃ | 20 | mg |
| Ca(NO₃)₂•4H₂O | 60 | mg |
| KCl | 10 | mg |
| MgSO₄•7H₂O | 20 | mg |
| Na₂glyceroPO₄ | 10 | mg |
| Na₂EDTA | 5 | mg |
| FeCl₃•6H₂O | 240 | μg |
| H₃BO₃ | 1 | mg |
| MnCl₂•4H₂O | 7.2 | mg |
| ZnCl₂ | 50 | μg |
| CoCl₂•6H₂O | 20 | μg |
| Tris amino | 100 | mg |
| Thiamin•HCl | 100 | μg |
| Biotin | 10 | μg |
| Vitamin B12 | 10 | μg |

TABLE 3
Composition of IMK medium

| | for 1 L | |
|---|---|---|
| NaNO₃ | 200 | mg |
| Na₂HPO₄ | 1.4 | mg |
| K₂HPO₄ | 5 | mg |
| NH₄Cl | 2.68 | mg |
| Fe-EDTA | 5.2 | mg |
| Mn-EDTA | 332 | μg |
| Na₂-EDTA | 37.2 | mg |
| ZnSO₄•7H₂O | 23 | μg |
| CoSO4•7H2O | 14 | μg |
| Na₂MoO₄•2H₂O | 7.3 | μg |
| CuSO₄•5H₂O | 2.5 | μg |
| H₂SeO₃ | 1.7 | μg |
| MnCl₂•4H₂O | 180 | μg |
| Thiamin•HCl | 200 | μg |
| Biotin | 1.5 | μg |
| Vitamin B12 | 1.5 | μg |
| Artificial sea water | 35.96 | g |

Sterilized culture tubes (16 mm×150 mm) (product of VWR) each plugged with a sponge stopper (60882-167, product of VWR) were used, and a sterilized medium (10 mL/tube) was dispensed to the tubes. Each alga strain (100 μL (in the case of liquid medium) or 1 platinum loop (in the case of solid medium)) was inoculated to the culture medium. Stationary culturing was performed at room temperature (22° C. to 24° C.) under a fluorescent lamp (illuminance: about 3,000 lux, illumination for 12 hours and dark for 12 hours).

Through centrifugation of the algae culture at 3,000 rpm for 30 minutes, an alga pellet was obtained. The alga pellet was dried at 80° C. for about 3 hours to about 16 hours, to thereby obtain dry algae, and the weight of the dry product was measured. The dry product was suspended in 1% saline (0.5 mL), and 5 mg/mL 7-pentadecanone (10 μL) was added as an internal standard to the suspension. Subsequently, chloroform (0.5 mL) and methanol (1 mL) were added to the suspension, and the mixture was vigorously stirred and then allowed to stand for 30 minutes. Thereafter, chloroform (0.5 mL) and 1.5% KCl (0.5 mL) were added to the mixture and stirred, followed by centrifugation at 3,000 rpm for 15 minutes. The formed chloroform layer (lower layer) was recovered.

The thus-prepared lipid fraction (about 500 μL) was treated with nitrogen to dryness, and 0.5 N potassium hydroxide/methanol solution (700 μL) was added to the dried fraction, and then incubated at 80° C. for 30 minutes. Subsequently, 14% boron trifluoride solution (product of SIGMA) (1 mL) was added to the fraction, and then incubated at 80° C. for 20 minutes. Then, hexane (1 mL) and saturated saline (1 mL) were added to the above mixture, and the mixture was allowed to stand at room temperature for 30 minutes. The thus-obtained hexane layer (upper layer) was recovered and analyzed by GC.

The GC analysis was performed under the following conditions: chromatograph, HP 7890A GC-FID (product of Agilent); column, DB-1 MS 30 m×200 μm×0.25 μm (product of J&W scientific); mobile phase, high-purity helium; flow rate, 1 mL/min; and temperature elevation, 100° C. (1 minute), 5° C./min, and 280° C. (20 minutes). As saturated fatty acid controls, the following commercial products (all produced from SIGMA) were purchased and analyzed: methyl laurate (C12), methyl myristate (C14), methyl palmitate (C16), and methyl stearate (C18). As unsaturated fatty acid controls, the following commercial products (all produced from SIGMA) were purchased and analyzed: methyl palmitoleate (C16:1), methyl oleate (C18:1), methyl linoleate (C18:2), methyl linolenate (C18:3), methyl eicosapentaenoate (C20:5), and methyl docosahexaenoate (C22:6). Identification of fatty acids was performed on the basis of coincidence in retention time between the fatty acid analyte and the corresponding standard. Lauric acid was also identified by GC-MS. C16 multi-unsaturated fatty acids were estimated from the GC-MS analytical results and are represented by C16:x (x is 2 or 3, wherein x represents the number of unsaturated bonds in fatty acid). The GC-MS analysis was performed under the following conditions: chromatograph, HP7890A GC and 5975C MS (products of Agilent); column, DB-1 ms 30 m×200 μm×0.25 μm (product of J&W scientific); mobile phase, high-purity helium; flow rate, 1 mL/min; and temperature elevation, 100° C. (1 minute), 5° C./min, and 280° C. (20 minutes). The amount of a fatty acid ester detected through GC analysis was calculated with reference to the internal standard, and the sum of the amounts of fatty acids was employed as the total fatty acid amount. The value obtained by dividing the total fatty acid amount by the amount of dry algae and multiplying the ratio by 100 was employed as a fatty acid content (%).

Table 4 shows the fatty acid compositional data of tested algae species.

TABLE 4
Fatty acid composition analysis

| | | Age | Productivity | Fatty acid composition (%) | | | | |
|---|---|---|---|---|---|---|---|---|
| Class | Algae No. | (days) | (mg/L) | C12:0 | C14:0 | C16:0 | C16:1 | C16:2 |
| Bacillariophyceae | LB 2658 | 18 | 89.3 | 0.0 | 7.3 | 41.7 | 34.1 | 0.0 |
| | 646 | 39 | 28.7 | 0.0 | 7.6 | 14.6 | 36.5 | 8.6 |
| | B 2085 | 39 | 26.0 | 0.0 | 5.9 | 25.3 | 28.1 | 5.0 |
| | B 2047 | 42 | 36.9 | 0.0 | 7.7 | 18.0 | 45.5 | 4.3 |
| | B 2049 | 42 | 65.7 | 0.0 | 4.3 | 21.2 | 29.2 | 9.0 |
| | LB 2611 | 21 | 38.3 | 0.0 | 7.2 | 23.7 | 60.0 | 2.6 |
| | 674 | 21 | 26.9 | 0.0 | 6.9 | 16.1 | 71.4 | 0.0 |
| | B 679 | 42 | 21.8 | 0.0 | 4.1 | 26.5 | 46.5 | 7.8 |
| Chlorophyceae | LB 835 | 7 | 15.1 | 0.0 | 0.0 | 26.6 | 0.0 | 0.0 |
| | LB 1970 | 19 | 19.5 | 0.0 | 0.0 | 26.6 | 0.0 | 0.0 |
| | LB 2544 | 48 | 25.3 | 0.0 | 1.5 | 32.5 | 3.1 | 0.0 |
| | LB 2068 | 59 | 25.6 | 0.0 | 0.6 | 17.3 | 8.1 | 0.0 |
| | 1246 | 44 | 5.1 | 0.0 | 0.0 | 27.3 | 0.0 | 0.0 |
| | 1783 | 44 | 58.4 | 0.0 | 0.0 | 23.0 | 0.0 | 0.0 |
| Cryptophyceae | LB 2423 | 69 | 43.8 | 0.0 | 7.7 | 34.9 | 38.1 | 4.5 |
| Cyanophyceae | LB 2061 | 21 | 9.0 | 0.0 | 0.0 | 67.0 | 0.0 | 0.0 |
| | LB 2425 | 56 | 24.1 | 0.0 | 0.0 | 42.2 | 6.8 | 0.0 |
| Phaeophyceae | LB 1426 | 69 | 18.3 | 0.0 | 4.8 | 23.7 | 0.0 | 0.0 |
| | LB 1636 | 69 | 40.3 | 0.0 | 3.5 | 18.9 | 0.0 | 0.0 |
| Xanthophyceae | LB 2067 | 68 | 19.9 | 0.0 | 6.9 | 30.2 | 60.2 | 0.0 |
| | 157 | 48 | 12.2 | 0.0 | 7.3 | 29.3 | 63.3 | 0.0 |

| | | Fatty acid composition (%) | | | | | | |
|---|---|---|---|---|---|---|---|---|
| Class | Algae No. | C16:3 | C18:0 | C18:1 | C18:2 | C18:3 | C20:5 | C22:6 |
| Bacillariophyceae | LB 2658 | 5.5 | 1.2 | 0.0 | 0.0 | 0.0 | 10.2 | 0.0 |
| | 646 | 0.0 | 0.0 | 0.0 | 0.0 | 0.0 | 32.8 | 0.0 |
| | B 2085 | 0.0 | 0.0 | 0.0 | 0.0 | 0.0 | 35.7 | 0.0 |
| | B 2047 | 0.0 | 0.0 | 2.1 | 1.5 | 0.0 | 20.9 | 0.0 |
| | B 2049 | 0.0 | 0.0 | 2.1 | 3.2 | 3.3 | 27.6 | 0.0 |
| | LB 2611 | 0.0 | 0.0 | 0.0 | 0.0 | 0.0 | 6.5 | 0.0 |
| | 674 | 0.0 | 0.0 | 0.0 | 0.0 | 0.0 | 5.6 | 0.0 |
| | B 679 | 0.0 | 0.0 | 2.7 | 0.0 | 0.0 | 12.3 | 0.0 |
| Chlorophyceae | LB 835 | 0.0 | 0.0 | 18.6 | 9.1 | 45.7 | 0.0 | 0.0 |
| | LB 1970 | 5.5 | 0.0 | 23.9 | 0.0 | 43.9 | 0.0 | 0.0 |
| | LB 2544 | 1.4 | 0.0 | 13.9 | 7.7 | 39.8 | 0.0 | 0.0 |
| | LB 2068 | 0.0 | 1.4 | 12.0 | 32.4 | 28.3 | 0.0 | 0.0 |
| | 1246 | 0.0 | 0.0 | 0.0 | 13.6 | 59.1 | 0.0 | 0.0 |
| | 1783 | 12.9 | 3.2 | 40.2 | 7.7 | 13.0 | 0.0 | 0.0 |
| Cryptophyceae | LB 2423 | 0.0 | 2.1 | 1.6 | 0.0 | 0.0 | 11.1 | 0.0 |
| Cyanophyceae | LB 2061 | 0.0 | 6.0 | 0.0 | 17.4 | 9.6 | 0.0 | 0.0 |
| | LB 2425 | 10.4 | 0.0 | 17.6 | 7.4 | 15.6 | 0.0 | 0.0 |
| Phaeophyceae | LB 1426 | 0.0 | 0.0 | 4.8 | 13.4 | 23.0 | 30.4 | 0.0 |
| | LB 1636 | 0.0 | 0.0 | 15.9 | 9.8 | 28.1 | 23.7 | 0.0 |
| Xanthophyceae | LB 2067 | 0.0 | 0.0 | 2.7 | 0.0 | 0.0 | 0.0 | 0.0 |
| | 157 | 0.0 | 0.0 | 0.0 | 0.0 | 0.0 | 0.0 | 0.0 |

The fatty acid composition of the lipids produced by various algae species was analyzed. However, no algae species in which lauric acid was accumulated was found.

### Example 1

A Zooxanthella microadriatica (in class Dinophyceae) strain LB2281 obtained from UTEX was employed in the experiment.

The microorganisms were cultured in a Daigo IMK medium. Sterilized culture tubes (16 mm×150 mm) (product of VWR) each plugged with a sponge stopper (60882-167, product of VWR) were used as culture containers, and a sterilized medium (10 mL/tube) was dispensed to the tubes. 100 μL of algal culture was inoculated to a new culture medium. Culturing was performed at room temperature (22° C. to 24° C.) under a fluorescent lamp (illuminance: about 3,000 lux, illumination for 12 hours and dark for 12 hours) for 59 days.

In a manner similar to that employed in Referential Example 1, recovery of algae, extraction of lipid, methyl esterification, and GC analysis were performed, to thereby analyze the fatty acid composition. Table 5 shows the results.

TABLE 5
Fatty acid composition of Z. microadriatica (strain LB2281)

| | | Age | Productivity | Fatty acid composition (%) | | | | | | |
|---|---|---|---|---|---|---|---|---|---|---|
| Class | Algae No. | (days) | (mg/L) | C12:0 | C14:0 | C16:0 | C18:0 | C18:1 | C18:2 | C22:6 |
| | LB 2281 | 59 | 43.4 | 13.2 | 19.1 | 38.1 | 1.9 | 9.4 | 1.8 | 16.6 |

In Zooxanthella microadriatica (strain LB2281), accumulation of lauric acid was observed that it content reached 13.2% of total fatty acid amount.

### Example 2

### Production of Algae Oil Having High Lauric Acid Content

An oil or fat having high lauric acid content was produced in the following manner.

Zooxanthella microadriatica (strain LB2281) was cultivated in a 500-mL Sakaguchi flask that contained 200 mL of IMK Medium, and stationary culturing was performed at room temperature (22° C. to 24° C.) under illumination (illuminance: about 3,000 lux, illumination for 12 hours and dark for 12 hours) for 38 days. The culture liquid was centrifuged at 3,000 rpm for 30 minutes, to thereby recover cells.

The alga was dried at 80° C. for about 16 hours, and chloroform/methanol (C/M) (1:1) (4 mL) was added to the dried algae. Under sonication, oil or fat was extracted at 40° C. for 30 minutes. In a similar manner, extraction with C/M (1:1) (4 mL) was performed again. The thus-recovered supernatant (about 8 mL) was filtered through a membrane filter (product of Millipore, Millex-LH, Hydrophilic PTFE, 0.45 μm, φ25 mm). The filtrate was dried by means of a centrifugal evaporator, to thereby obtain 19.4 mg of an oil or fat fraction of the strain LB2281.

Through a method similar to that described in Example 1, the oil or fat composition was analyzed. The fatty acid content of the oil or fat fraction was 34.4%, and the lauric acid content of the total fatty acid amount was 7.8%. That is, lauric acid (0.518 mg) was recovered from the oil or fat fraction (19.4 mg).

### Example 3

### Accumulation of C12 in Dinophyceae

A Zooxanthella microadriatica (in class Dinophyceae) strain LB2282 obtained from UTEX and Symbiodinium sp. CCMP2948 obtained from the Provasoli-Guillard National Center for Culture of Marine Phytoplankton (CCMP) were employed in the experiment.

The alga was cultured in a Daigo IMK medium. Sterilized culture tubes (16 mm×150 mm) (product of VWR) each plugged with a sponge stopper (60882-167, product of VWR) were used, and a sterilized medium (10 mL/tube) was dispensed to the tubes. 100 μL of each alga culture was inoculated to the culture medium. Culturing was performed at room temperature (20° C.) under a fluorescent lamp (illuminance: about 3,000 lux, illumination for 12 hours and dark for 12 hours) for about two months.

In a manner similar to that employed in Referential Example 1, recovery of algae, extraction of lipid, methyl esterification, and GC analysis were performed, to thereby analyze the fatty acid composition. Table 6 shows the results.

TABLE 6
Fatty acid composition of genus Symbiodinium (Zooxanthella)

| | | | | Produc- | | | | | | | | | |
|---|---|---|---|---|---|---|---|---|---|---|---|---|---|
| | | | Age | tivity | Fatty acid composition(%) | | | | | | | | |
| Class | No. | Name | (days) | (mg/L) | C12:0 | C14:0 | C14:1 | C16:0 | C16:1 | C18:0 | C18:1 | C18:2 | C22:6 |
| Dinophyceae | LB 2282 | Z. | 56 | 13.06 | 3.5 | 6.2 | 2.8 | 54.7 | 9.6 | 5.9 | 3.5 | 1.1 | 12.6 |
| | | microadriatica | | | | | | | | | | | |
| | CCMP2948 | Symbiodinium | 61 | 22.90 | 5.5 | 6.5 | 0.6 | 45.6 | 6.9 | 8.0 | 6.7 | 1.2 | 19.2 |
| | | sp. | | | | | | | | | | | |

In genus Symbiodinium (Zooxanthella), accumulation of lauric acid was observed with 3% or higher of the total fatty acid amount.

1. A method for producing an oil or fat containing lauric acid as a constituent fatty acid, which method comprises culturing an alga belonging to the genus Symbiodinium in a medium and recovering, from the culture product, an oil or fat having a lauric acid content of 3 weight % or higher of the fatty acid composition.

2. A method according to claim 1, wherein the alga belonging to the genus Symbiodinium is Symbiodinium microadriaticum.

3. A method according to claim 2, wherein the alga belonging to the genus Symbiodinium is a Zooxanthella microadriatica strain LB2281, or an alga strain having virtually the same mycological properties as those of strain LB2281.

4. A method according to any one of claims 1 to 3, wherein culturing is performed for 7 to 120 days under light irradiation at an illuminance of 300 to 10,000 lux.

5. A method for producing lauric acid, wherein the method comprises separating and recovering lauric acid from an oil or fat produced through a method as recited in claim 1.
